(19) United States
(12) Patent Application Publication
(10) Pub. No.: US 2024/0099876 A1
(43) Pub. Date: Mar. 28, 2024

(54) OSTOMY COLLECTION DEVICE

(71) Applicant: Salts Healthcare Limited, Birmingham (GB)

(72) Inventors: Marcus Allen, Telford (GB); Mark Heynes, Newcastle-under-Lyme (GB); Danny Morris, Stafford (GB); Phillip SAMPEY, Alsager (GB)

(21) Appl. No.: 18/264,341

(22) PCT Filed: Feb. 3, 2022
(86) PCT No.: PCT/GB2022/050293

§ 371 Date: Aug. 4, 2023

(30) Foreign Application Priority Data
Feb. 5, 2021 (GB) 2101654.8

(51) Int. Cl. A61F 5/445; A61F 5/44

(52) U.S. Cl. CPC A61F 5/445; A61F 5/4404

(57) ABSTRACT

An ostomy collection device (10) including: a housing (12) which defines a collection volume for holding waste, and a connector member (30) for connection to an ostomy appliance, having an inlet (40) and an outlet (42) fluidly connected to the collection volume, said connector member (30) being pivotably and/or rotatably supported relative to the housing (12).

### DESCRIPTION OF INVENTION

Embodiments of the present invention relate to an ostomy collection device.

Ostomy appliances are well known. Typically, a person having a stoma uses an ostomy appliance to collect waste from their stoma. The ostomy appliances collects a volume of waste and, once full or nearing full, the user either changes the appliance for a fresh one or empties the waste from the existing appliance.

This process works well while the user is able to visit a suitable space for changing/emptying their ostomy appliance—e.g. a bathroom or toilet. However, there may be times when the user does not wish to empty or change their appliance as often, for example, when travelling or while asleep.

In such times, it would be beneficial to provide an additional collecting volume that is connected to the existing ostomy appliance, so that the appliance does not need to be emptied as often. This functionality is currently provided by a flexible bag which is connected to the ostomy appliance.

Alternative systems are sought for ostomy appliance users. Embodiments of the present invention seeks to alleviate one or more drawbacks of the prior art.

Embodiments of the present invention are described, by way of example only, with reference to the accompanying drawings, in which:

According to an aspect of the invention we provide an ostomy collection device including: a housing which defines a collection volume for holding waste, and a connector member for connection to an ostomy appliance, having an inlet and an outlet fluidly connected to the collection volume, said connector member being pivotably and/or rotatably supported relative to the housing.

Further optional features of the ostomy collection device are provided in the appended claims.

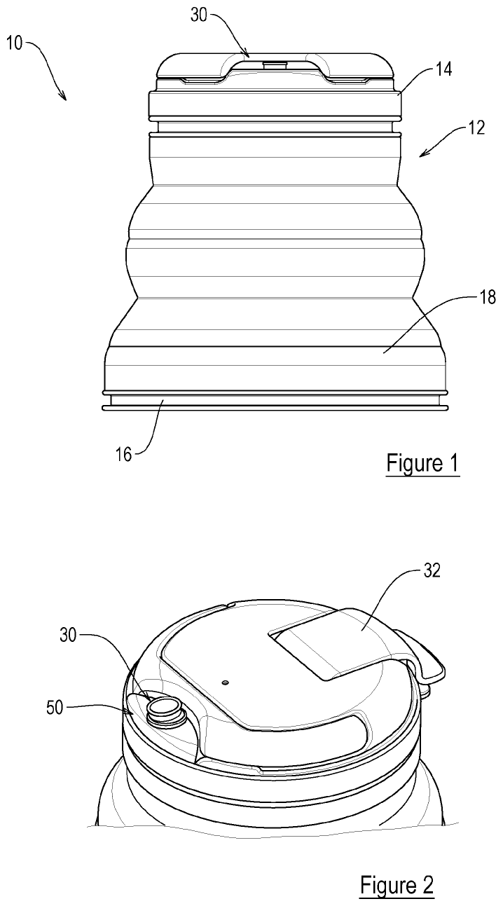

FIG. 1 is a side view of an ostomy collection device,

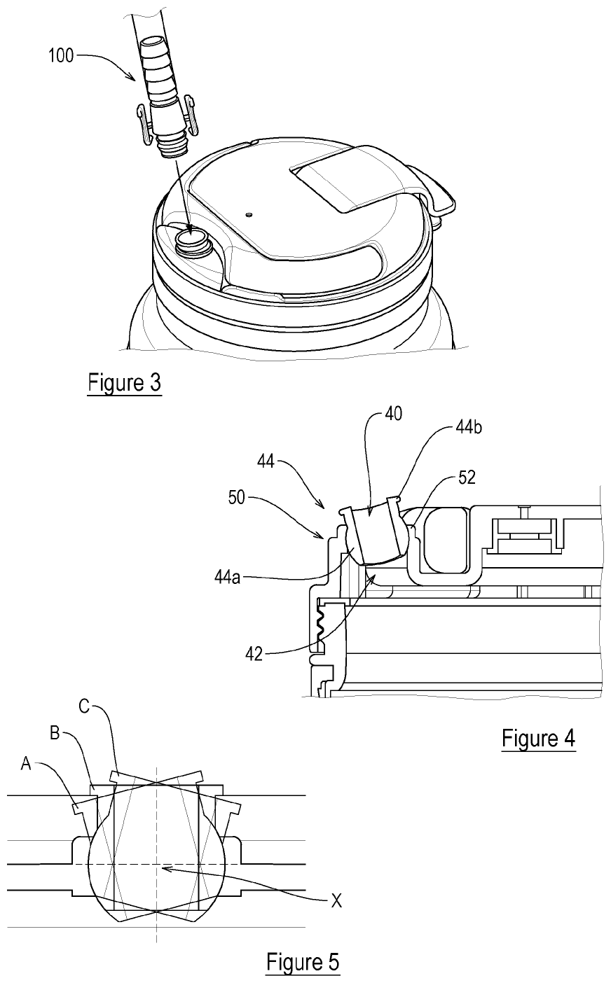

FIGS. 2 and 3 are perspective views of a top of an ostomy collection device,

FIGS. 4 and 5 show cross-sectional view of a part of a top of an ostomy collection device,

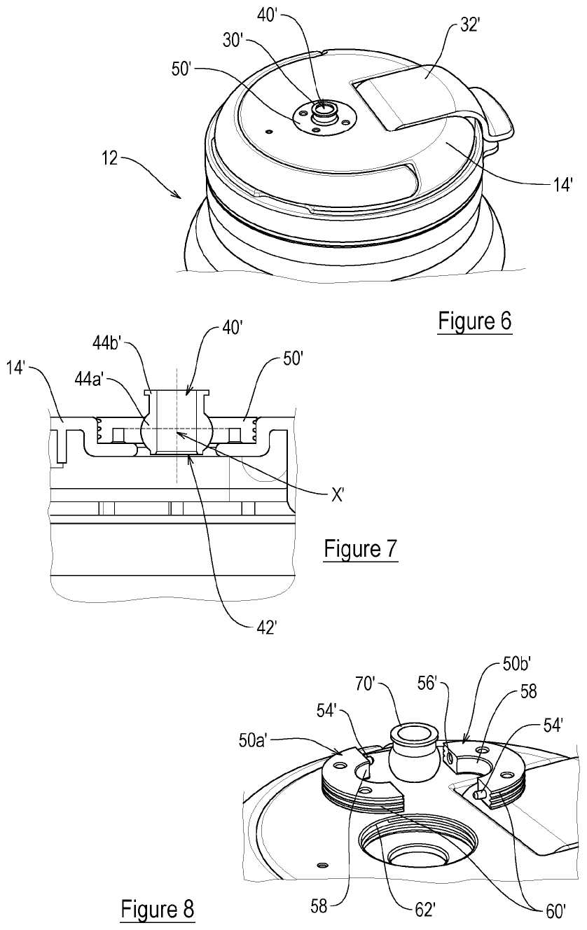

FIG. 6 is a perspective view of a further embodiment of top of an ostomy collection device,

FIG. 7 shows a cross-sectional view of the top of the ostomy collection device, and

FIG. 8 is a blown up view of the top of the ostomy collection device.

An ostomy collection device 10 is shown in FIG. 1. The ostomy collection device 10 is configured to connect to an outlet or a tube connected to the outlet of a user's ostomy appliance that is connected about their stoma (and collecting waste exiting the stoma). The ostomy collection device 10 is particularly suited to urostomy use where the waste being collected is more fluid that other ostomy waste outputs and can, therefore, be emptied easily from the collection vessel into a toilet or waste disposal.

The ostomy collection device 10 includes a housing 12, which defines a collecting volume for receiving and storing waste from an ostomy appliance. The ostomy collection device 10 also includes a connector member 30 and preferably an outlet.

The connector member 30 connects to an ostomy appliance (the outlet of the ostomy appliance or an additional tube may connect to the appliance outlet and extend to the ostomy collection device 10). The tube includes connector (an example of which is shown by connector 100 in FIG. 3) on the end, to attach to the connector member 30 of the ostomy collection device 10.

The connector member 30 has an inlet 40 and an outlet 42, which are connected by a flow path that communicates with the collection volume. The connector member 30 is pivotably and/or rotatably supported relative to the housing 12. In more detail, the connector member 30 is held in a formation such that it is able to pivot/move/rotate about a substantially central point without losing connection with the housing (i.e. without shutting the connection or flow path through to the collection volume). This is advantageous because the permitted movement of the connection member 30 reduces the chance that the connection to the ostomy appliance will destabilise the ostomy collection device 10 (and potentially cause it to fall over).

The connector member 30 is held so that is rotates about a central point (illustrated as point “X” in FIG. 5). In other words, the connector member 30 has no translational degrees of freedom (when in normal use) but does have three rotational degrees of freedom. The connector member 30 has a central axis (i.e. an axis that extends through “X”) and, in this example, the axis passes through the inlet 40 and the outlet 42. As the connector member 30 moves relative to the housing, the orientation/angle of the axis changes relative to the housing 12.

In some embodiments, the connector member 30 has a body 44 which has a spherical/rounded part 44a and a formation 44b for attaching to the ostomy appliance. Three different positions are illustrated in FIG. 5 to show the general principle—the spherical/rounded part 44a of the body 44 is always located in the same translational position and held in place by the housing 12.

However, the formation 44b for attaching to the ostomy appliance has multiple positions at different angles (FIG. 5 has a translucent indication of each position—referenced with A, B and C to show where they are explicitly).

In some embodiments, a support formation 50 engages the connector member and supports it in position so that the connector member 30 is able to move as discussed. The support formation 50 also includes an opening that corresponds with the outlet of the connector member 30, so that the flow path between the inlet 40, the outlet 42 to the collection volume is maintained.

The support formation 50 provides a recessed receiving part that acts as a socket. The spherical part 44a of the connector member 30 acts as a “ball” part and is received by the support formation 50 to form a ball and socket joint/connection. It should be appreciated that both the ball part and the socket part have respective openings to provide the flow path from the inlet of the connector member 30 to the collection volume.

In some embodiments, the support formation 50 includes a retaining portion 52. In the illustrated example (for example, in FIGS. 4 and 5), the retaining portion is a narrowing of the recessed receiving part which inhibits or prevents the connector member 30 from being removed from the support formation 50. In other words, an entrance of the support formation 50 is narrower which retains the body 44 of the connector member 30 in position.

In some embodiments, the retaining portion 52 provides a snap-fit for the connection connector member 30. In other words, the widest part of the body 44 is wider than the entrance to the receiving part of the support formation 50, so once the connector member 30 is pushed/forced into the support formation 50 during manufacture, the connector member 30 does not fall out/disconnect from the housing 12.

In some embodiments, the support formation 50 is formed integrally with at least one other part of the housing 12. In embodiments (such as illustrated in FIGS. 2 to 4), the housing 12 includes a top 14 and a base 16 connected by a wall 18. In such an example, the support formation 50 is formed as part of the top 14 of the housing 12.

Another embodiment is illustrated in FIGS. 6 to 8. Where features are the same as those already described above in relation to the first embodiment, the reference number used is the same and a prim symbol is included—in other words, the ostomy collection device 10 will become ostomy collection device 10′.

FIGS. 6 to 8 illustrate various views of a top 14′ of a housing 12′ which includes a connector member 30′ and support formation 50′. In embodiments, the support formation 50′ is separate from the housing 12′. In other words, the support formation 50′ can be removed from the housing 12′.

The support formation 50′ includes an engagement formation 60′, which is configured to engage with a corresponding portion/engagement formation 62′ of the housing 12′. In some embodiments, the engagement formation 60′ and the corresponding portion/engagement formation 62′ include a threaded portion. Thus, the support formation 50′ is screwed into the housing 12′ (and held securely in position).

In some embodiments, the housing 12′ includes an opening that at least partially aligns with the outlet 42′ of the connector member 30′ — this ensures that the waste flow path is maintained into the collection volume of the ostomy collection device 10′ In some embodiments, the support formation 50′ is formed from two separate parts—a first support piece 50a′ and a second support piece 50b′. The connector member 30′ is a similar shape to that already described above—i.e. has a body 44′ with a spherical/rounded part 44a′ and a formation 44b′ to connect to an ostomy appliance. The first and second support pieces 50a′, 50b′ extend around the body 44′ of the connector member 30′ and engage with one another when in position. The first and second support pieces 50a′, 50b′ extend around an outer surface of the connector member 30′.

This allows the support formation 50′ to provide a retaining portion (which functions to prevent or inhibit the connector member 30′ from moving from the support formation 50′) without relying on a snap fit. An advantage of this may be that distortion or weakening of the material that could occur with a snap fit fitting if repeated many times is avoided.

In embodiments, the first and second support piece 50a′, 50b′ are both C-shaped in top down profile and come together to form an entire ring/annulus around the connector member 30′. In other words, when viewed from above, both support pieces 50a′, 50b′ are the same half annular shape. It should be appreciated that as long as the pieces of the support formation 50′ come together to form an entire ring (to extend around the connector member 30′) the support pieces may form different portions of the annulus.

In embodiments, an inward facing surface 58 on the support pieces 50a′, 50b′ is concave (when viewed as a cross-section). In other words, the surface that contacts the connector member 30′ is curved to echo the contour of the spherical part 44a′ of the connector member 30′.

In embodiments, the first support piece 50a′ has a first engagement formation and the second support piece 50b′ has a second engagement formation. These engagement formations cooperate when the first support piece 50a′ and the second support piece 50b′ are brought together to form the whole support formation 50′. In other words, the engagement formations hold the first and second support pieces 50a′, 50b′ in a desired position.

In embodiments, the first and the second engagement formation include at least one of a projection 54′ and an opening 56′ (where the formations cooperate by the projection 54′ being received in the opening 56′). In the illustrated example, both the first and second engagement formations include a projection 54′ and an opening 56′ (note the opening 56′ on the second support piece cannot be seen in FIG. 8).

In the illustrated examples, the formation 44b, 44b′ provides an attachment zone for an ostomy appliance or a tube from an ostomy appliance to be connected. An example connector 100 is illustrated in FIG. 3 and it should be appreciated that this type of connector may cooperate with many designs of connector member 30, 30′. In some embodiments, the formation 44b, 44b′ includes a ridge or flange, which cooperates with a connector attached to an ostomy appliance to provide a substantially sealed/water tight connection with the ostomy collection device 10, 10′.

Thus, the connector member 30, 30′ provides a connection to the ostomy appliance/tube connected to the ostomy appliance that allows the relative angle between the appliance and collection device 10, 10′ to change (i.e. the tube connected to the connector member 30, 30′ is able to pivot/rotate/change angle with respect to the collection device 10, 10′). In other words, the connector member 30, 30′ is supported by the housing 12, 12′/the support formation 50, 50′ so that is provides a pivoting connection between the ostomy collection device 10, 10′ and the ostomy appliance/tube.

The ostomy collection device includes an outlet, which is configured for emptying the contents of the collecting volume (e.g. into a toilet, etc. so that the waste can be disposed of). A lid portion 32 covers the outlet and allows the outlet to be opened and closed, as desired by the user.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An ostomy collection device including:
a housing which defines a collection volume for holding waste, and
a connector member for connection to an ostomy appliance, having an inlet and an outlet fluidly connected to the collection volume, said connector member being pivotably and/or rotatably supported relative to the housing.

2. The ostomy collection device according to claim 1, wherein a support formation engages and supports the connector member.

3. The ostomy collection device according to claim 2, wherein the connector member forms a substantially spherical or round part and the support formation forms a socket part that receives the round part.

4. The ostomy collection device according to claim 1, wherein the support formation includes a retaining portion which inhibits or prevents the connector member from being removed from the support formation.

5. The ostomy collection device according to claim 4, wherein the retaining portion provides a snap-fit when the connector member is pushed into the support formation.

6. The ostomy collection device according to claim 2, wherein the support formation is formed integrally with the housing.

7. The ostomy collection device according to claim 2, wherein the support formation is separate from the housing.

8. The ostomy collection device according to claim 7, wherein the support formation includes an engagement portion which is configured to engage with a corresponding portion of the housing.

9. The ostomy collection device according to claim 8, wherein the engagement portion and the corresponding portion are threaded.

10. The ostomy collection device according to claim 7, wherein the support formation is formed from a first and second support piece.

11. The ostomy collection device according to claim 10, wherein the first and second support piece extend around an outer surface of the connector member.

12. The ostomy collection device according to claim 10, wherein the first support piece has a first engagement formation and the second support piece has a second engagement formation, such that when the first and second support piece are in position the first and second engagement formations interact to assist holding the first and second support pieces in place.

13. The ostomy collection device according to claim 12, wherein the first and/or second engagement formations of the respective first and second support piece include at least one projection and at least one recess.

14. The ostomy collection device according to claim 1, wherein the connector member includes an attachment formation for connecting to an ostomy appliance.

15. The ostomy collection device according to claim 14, wherein the attachment formation includes a ridge or a flange.

16. The ostomy collection device according to claim 1, wherein the connector member is supported relative to the housing and has zero degrees of translational freedom.

17. The ostomy collection device according to claim 1, wherein the connector member provides a pivoting and/or rotating connection between the collection device and a tube or a conduit which is connected to the connector member.

18. A connector member for an ostomy collection device according to claim 1.
